(19) United States
(12) Patent Application Publication
(10) Pub. No.: US 2022/0304846 A1
(43) Pub. Date: Sep. 29, 2022

(54) Vaginal Shield Assembly

(71) Applicants: Deborah Mitchell, Mobile, AL (US); Fred Mitchell, Mobile, AL (US)

(72) Inventors: Deborah Mitchell, Mobile, AL (US); Fred Mitchell, Mobile, AL (US)

(21) Appl. No.: 17/210,592

(22) Filed: Mar. 24, 2021

(51) Int. Cl. A61F 5/455

(52) U.S. Cl. CPC A61F 5/4556

(57) ABSTRACT

A vaginal shield assembly for protecting female genitalia from urine includes a panel that has a plurality of intersecting sides such that the panel has a triangular shape for positioning over female genitalia. The panel has a urine slot extending through the panel that is aligned with a urethra when the panel is positioned over the female genitalia. In this way the urine can pass through the urine slot. The panel is comprised of a fluid impermeable material to inhibit the urine from contacting the female genitalia. In this way the panel inhibits irritation that would develop from the urine contacting the female genitalia.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to shield devices and more particularly pertains to a new shield device for protecting female genitalia from urine. The device includes a triangular panel that has a urine slot passing through the panel. The panel can be positioned over female genitalia to inhibit urine from contacting the female genitalia.

### (2) Description of Related Art Including Information Disclosed Under 37 CFR 1.97 and 1.98

The prior art relates to shield devices including a variety of urinating devices that are positionable against female genitalia for inhibiting urine from contacting the female genitalia during urination. The prior art also discloses a variety of chute devices for directing the flow of urine from a female urethra. In no instance does the prior art disclose a panel that is positionable against female genitalia and which has a urine slot therein for passing urine through the panel thereby inhibiting the urine from contacting the female genitalia.

### BRIEF SUMMARY OF THE INVENTION

An embodiment of the disclosure meets the needs presented above by generally comprising a panel that has a plurality of intersecting sides such that the panel has a triangular shape for positioning over female genitalia. The panel has a urine slot extending through the panel that is aligned with a urethra when the panel is positioned over the female genitalia. In this way the urine can pass through the urine slot. The panel is comprised of a fluid impermeable material to inhibit the urine from contacting the female genitalia. In this way the panel inhibits irritation that would develop from the urine contacting the female genitalia.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a vaginal shield assembly according to an embodiment of the disclosure.

FIG. 2 is a bottom view of an embodiment of the disclosure.

FIG. 3 is a front view of an embodiment of the disclosure.

FIG. 4 is a perspective in-use view of an embodiment of the disclosure.

### DETAILED DESCRIPTION OF THE INVENTION

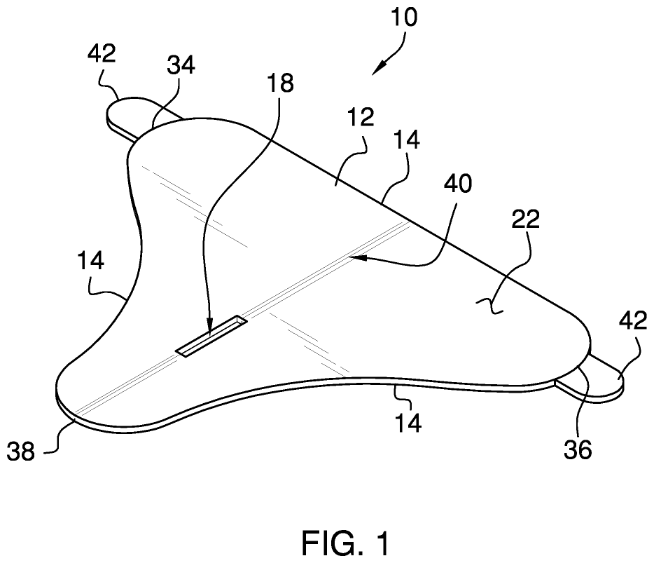

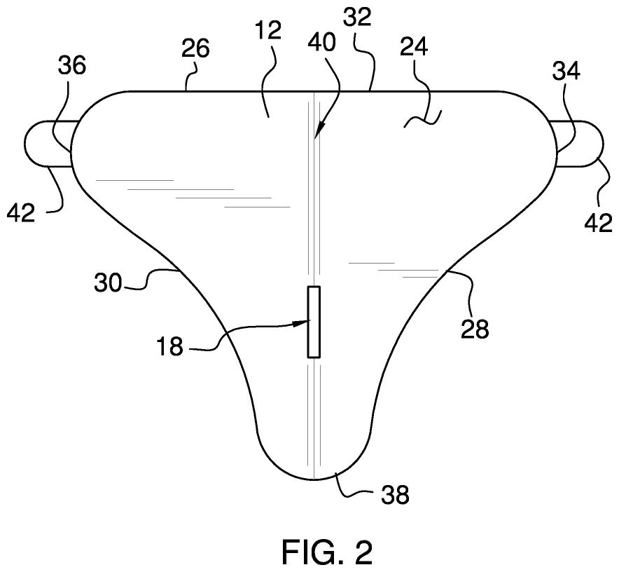

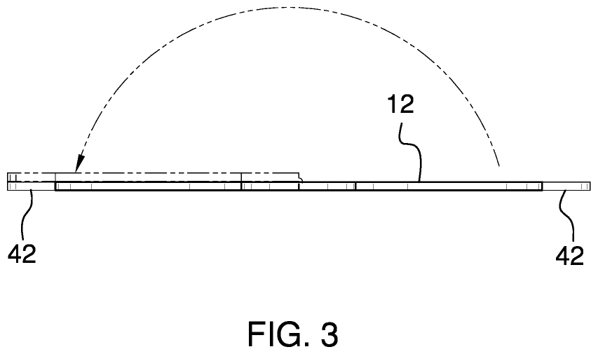

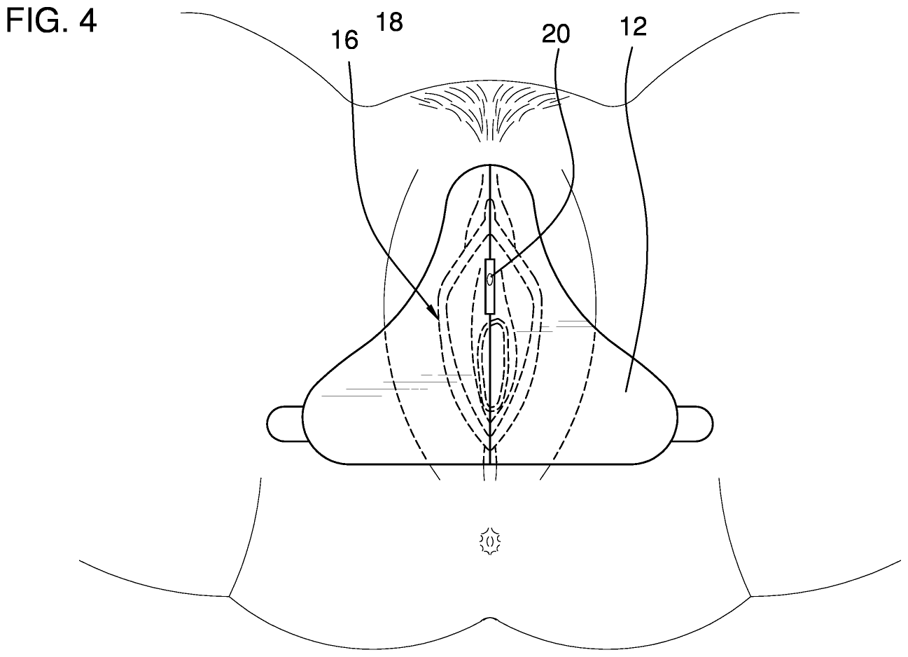

With reference now to the drawings, and in particular to FIGS. 1 through 4 thereof, a new shield device embodying the principles and concepts of an embodiment of the disclosure and generally designated by the reference numeral 10 will be described.

As best illustrated in FIGS. 1 through 4, the vaginal shield assembly 10 generally comprises a panel 12 that has a plurality of intersecting sides 14 such that the panel 12 has a triangular shape. In this way the panel 12 is shaped to facilitate the panel 12 to be positioned over female genitalia 16. The panel 12 has a urine slot 18 extending through the panel 12 and the urine slot 18 is aligned with a urethra 20 when the panel 12 is positioned over the female genitalia 16. In this way urine can pass through the urine slot 18 when the panel 12 is positioned on the female genitalia 16. Additionally, the panel 12 is comprised of a fluid impermeable material to inhibit the urine from contacting the female genitalia 16. In this way the panel 12 inhibits irritation that would develop from the urine contacting the female genitalia 16.

The panel 12 has a first surface 22, a second surface 24 and a perimeter edge 26 extending therebetween, and the perimeter edge 26 has a first side 28, a second side 30 and a third side 32. Each of the first side 28 and the second side 30 intersects the third side 32 at a respective one of a first corner 34 and a second corner 36 of the panel 12. Additionally, each of the first side 28 and the second side 30 intersect each other at a third corner 38, thereby facilitating the panel 12 to define the triangular shape. The panel 12 has a fold line 40 extending between the third side 32 and the third corner 38 such that the fold line 40 bisects the panel 12. The panel 12 is foldable along the fold line 40 into a closed position for storage having the first corner 34 being aligned with the second corner 36.

The urine slot 18 extends through the first surface 22 and the second surface 24 of the panel 12. Additionally, the urine slot 18 is positioned on the fold line 40 and the urine slot 18 is positioned closer to the third corner 38 than the third side 32. Each of the first corner 34, the second corner 36 and the third corner 38 is rounded. The first side 28 curves inwardly toward the fold line 40 between the first corner 34 and the third corner 38. The second side 30 curves inwardly toward the fold line 40 between the second corner 36 and the third corner 38. Thus, each of the first side 28 and the second side 30 accommodate the user's thighs when the panel 12 is positioned over the female genitalia 16.

A pair of tabs 42 is each coupled to and extends away from the panel 12. In this way each of the tabs 42 can be gripped by a user for positioning the panel 12 over the female genitalia 16. Each of the tabs 42 is coupled to the perimeter edge 26 of panel 12 and each of the tabs 42 is positioned on a respective one of the first corner 34 and the second corner 36.

In use, the panel 12 is positioned over the female genitalia 16 when the user has a bacterial infection, such as a yeast infection, that causes irritation on the female genitalia 16. The panel 12 is oriented such that the third corner 38 is directed upwardly and the urine slot 18 is aligned with the urethra 20. In this way urine passes through the urine slot 18 and the panel 12 inhibits the urine from contacting the female genitalia 16. Thus, the urine is inhibited from causing burning and discomfort that would result from the urine contacting the site of the bacterial infection on the female genitalia 16. The panel 12 can be rinsed or washed after use to facilitate the panel 12 to be used repeatedly.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A vaginal shield assembly for inhibiting urine from contacting irritated skin during urination, said assembly comprising:
a panel having a plurality of intersecting sides such that said panel has a triangular shape wherein said panel is configured to be positioned over female genitalia, said panel having a urine slot extending through said panel wherein said urine slot is configured to be aligned with a urethra when said panel is positioned over the female genitalia thereby facilitating urine to pass through said urine slot, said panel being comprised of a fluid impermeable material wherein said panel is configured to inhibit the urine from contacting the female genitalia thereby inhibiting irritation that would develop from the urine contacting the female genitalia.

2. The assembly according to claim 1, wherein said panel has a first surface, a second surface and a perimeter edge extending therebetween, said perimeter edge having a first side, a second side and a third side, each of said first side and said second side intersecting said third side at a respective one of a first corner and a second corner of said panel, each of said first side and said second side intersecting each other at a third corner, said panel having a fold line extending between said third side and said third corner such that said fold line bisects said panel, said panel being foldable along said fold line into a closed position for storage having said first corner being aligned with said second corner.

3. The assembly according to claim 2, wherein said urine slot extends through said first surface and said second surface of said panel, said urine slot being positioned on said fold line, said urine slot being positioned closer to said third corner than said third side.

4. The assembly according to claim 2, wherein each of said first corner, said second corner and said third corner is rounded, said first side curving inwardly toward said fold line between said first corner and said third corner, said second side curving inwardly toward said fold line between said second corner and said third corner.

5. The assembly according to claim 2, further comprising a pair of tabs, each of said tabs being coupled to and extending away from said panel wherein each of said tabs is configured to be gripped by a user for positioning said panel over the female genitalia, each of said tabs being coupled to said perimeter edge of panel, each of said tabs being positioned on a respective one of said first corner and said second corner.

6. A vaginal shield assembly for inhibiting urine from contacting irritated skin during urination, said assembly comprising:
a panel having a plurality of intersecting sides such that said panel has a triangular shape wherein said panel is configured to be positioned over female genitalia, said panel having a urine slot extending through said panel wherein said urine slot is configured to be aligned with a urethra when said panel is positioned over the female genitalia thereby facilitating urine to pass through said urine slot, said panel being comprised of a fluid impermeable material wherein said panel is configured to inhibit the urine from contacting the female genitalia thereby inhibiting irritation that would develop from the urine contacting the female genitalia, said panel having a first surface, a second surface and a perimeter edge extending therebetween, said perimeter edge having a first side, a second side and a third side, each of said first side and said second side intersecting said third side at a respective one of a first corner and a second corner of said panel, each of said first side and said second side intersecting each other at a third corner, said panel having a fold line extending between said third side and said third corner such that said fold line bisects said panel, said panel being foldable along said fold line into a closed position for storage having said first corner being aligned with said second corner, said urine slot extending through said first surface and said second surface of said panel, said urine slot being positioned on said fold line, said urine slot being positioned closer to said third corner than said third side, each of said first corner, said second corner and said third corner being rounded, said first side curving inwardly toward said fold line between said first corner and said third corner, said second side curving inwardly toward said fold line between said second corner and said third corner; and
a pair of tabs, each of said tabs being coupled to and extending away from said panel wherein each of said tabs is configured to be gripped by a user for positioning said panel over the female genitalia, each of said tabs being coupled to said perimeter edge of panel, each of said tabs being positioned on a respective one of said first corner and said second corner.
